(19) United States
(12) Patent Application Publication
(10) Pub. No.: US 2002/0165267 A1
(43) Pub. Date: Nov. 7, 2002

(54) Phloxin B a color additive in cosmetics and food use thereof as anti-microbial agent.

(76) Inventor: Reuven Rasooly, Davis, CA (US)

Correspondence Address: RASOOLY REUVEN, 1722 RAINTREE PLACE, Davis, CA, 95616, US

(21) Appl. No.: 09/849,103

(22) Filed: May 4, 2001

(51) Int. Cl. A61K 31/353

(52) U.S. Cl. 514/454

(57) ABSTRACT

The invention provides use of Phloxin B, a color additive registered for human, permitted in the United States for use in coloring cosmetics and ingested drugs and coloring food in other countries, including Japan, as anti-microbial agent.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

Bacterial resistance to antimicrobial agents, such as beta-lactam, macrolides, quinolones, and vacomycin, is becoming a major worldwide health problem (1,2). There is an urgent need to discover new agents to effectively treat patients infected with multidrug-resistant bacteria. The present invention describes the use of one compound, Phloxine B, as a new antibacterial agent.

Bacillus spp. are frequently found in very wide range of foods and considered a significant problem to the food industry (84,000 cases of food poisoning in the USA each year). Food poisoning outbreaks involving Bacillus spp hare resulted in major product recalls and substantial economic loss. On the basis of genetic evidence, Bacillus anthracis, B. thuringiensis, very closely related (3). Bacillus anthracis causes the acute fatal disease anthrax, and has the potential of being used in biological weapons of mass destruction against civilian populations (4, 5). The dye Phloxine B (2′,4′,5′,7′-tetrachlorofluorecein disodium salt; C₂₀H₂Br₄Cl₄O₅Na₂), also referred to as D&C red No. 28, is a color additive registered in the United States for use in coloring cosmetics and ingested drugs (6). The FDA has set the acceptable daily intake (ADI) for humans at 1.25 mg/kg (7). Phloxine B is also used as a food colorant in other countries, including Japan (8).

Using Phloxin B, a coloring food dye that has very fast antibiotic activity, can reduce Bacillus Diarrhoeal food poisoning and reduce the threat of anthrax as a biological weapon of mass destruction. The present invention describes the potential use of Phloxine B for the treatment of bacterial infections.

### REFERENCES

1. Cohen, “Antimicrobial resistance: prognosis for public health” trends in microbiology, 2 (1994), 422-425.

2. C. M. Henry, Antibiotic resistance. Chemical and Engineering News, March 6 (2000), 41-58.

3. E. Helgason, et al., Applied and Environmental Microbiology, 66 (2000), 2627-2630.

4. M. Hugh-Jones, 1996-97 Global Anthrax Report. J. Appl. Micorbiol. 87 (1999), 189-191.

5. T. V. Inglesby, D. A. Henderson, J. G. Bartlett et al., Anthrax as a biological weapon: medical and public health management. J. Am. Med. Assoc. 281 (1999), 1735-1745.

6. Code of Federal Regulations, Title 21, Part 74.1328, US Government Printing Office, Washington, D.C., 1999.

7. Federal Register, 47 (1982), 42566-42569.

8. J. R. Heitz, Pesticidal application of halogenated xanthene dyes. Phytoparasitica 25 (1997), 89-92.

### BRIEF SUMMARY OF THE INVENTION

The invention provides use of Phloxin B, a color additive registered for human, permitted in the United States for use in coloring cosmetics and ingested drugs and coloring food in other countries, including Japan, as anti-microbial agent.

### DETAILED DESCRIPTION OF THE INVENTION

Phloxine B has very good antibiotic activity on bacteria such as B. mycoides, B. thuringiensis, and B. subtilis. This registered food dye decreased the bacterial count by 3 logs (from 10⁻⁷ to 10⁻⁴) in minutes after the dye was added at 100 μg/ml. Therapeutic effectiveness of antibiotics is based on a successful clinical outcome, and does not require that the antimicrobial agents kill 100% of the organisms involved in the infection. Frequently reducing organism loas by one log (factor of 10) allows time for the host's immune system to activate and control the infection.

### EXAMPLE 1

Sensitivity of bacteria to the commercially available Phloxine B measured by agar diffusion test. Principle of this microbiological assay: The bacteria were grown on Brain Heart Infusion (BHI) broth. Growth was analyzed by measuring turbidity with a spectrophotometer. During the logarithmic phase (OD of 0 3 at 600 nm), 200 μl of the bacterial culture was spread over the surface of an agar plate with a bent glass rod. Fifty μl of various concentrations of Phloxine B (200, 100, 50, 25, and 12.5 μg/ml diluted in the BHI broth) were added to the wells. The plate was then transferred to an incubator at 37° C. the appearance of clear zones around the wells after 16 hours, indicating inhibition of bacterial growth, was noted. Using this method, a Phloxine B concentration of 100 μg/ml resulted in clear zones of a size similar to those observed with tetracycline at 25 μg/ml and with half the inhibition zones observed with chloramphenicol at 200 μg/ml. The results of the experiment are shown in FIGS. 1 and 2.

### EXAMPLE 2

Sensitivity of the bacteria to Phloxine B measured by spectrophotometry and Colony-Forming Units. Principle of this microbiological assay: Various concentrations of Phloxine B (100, 75, 50, 25 μg/ml) were added to bacterial cultures in BHI broth during the logarithmic growth phase (OD of 0.3 at 600 nm). The survival rate was measured by plating serial diluations of the culture media during the time course on BHI plates, and counting the number of colonies. This count is expressed as Colony-Forming Units. Phloxine B inhbits bacterial growth in a dose-dependent manner. At 100 μg/ml, Phloxine B reduced the number of Colony-Forming Units by more than two logs (factor of 100). The results of the experiment are shown in FIGS. 3, 4 and 5.

### BRIEF DESCRIPTION OF THE FIGURES

FIG. 1. Agar diffusion method to compare inhibition growth of Bacillus thuringiensis ATCC55055 by antibiotic: chloramphenicol, tetracycline and various concentration of Phloxin B (200, 100, 50, 25, 12.5 μg/ml).

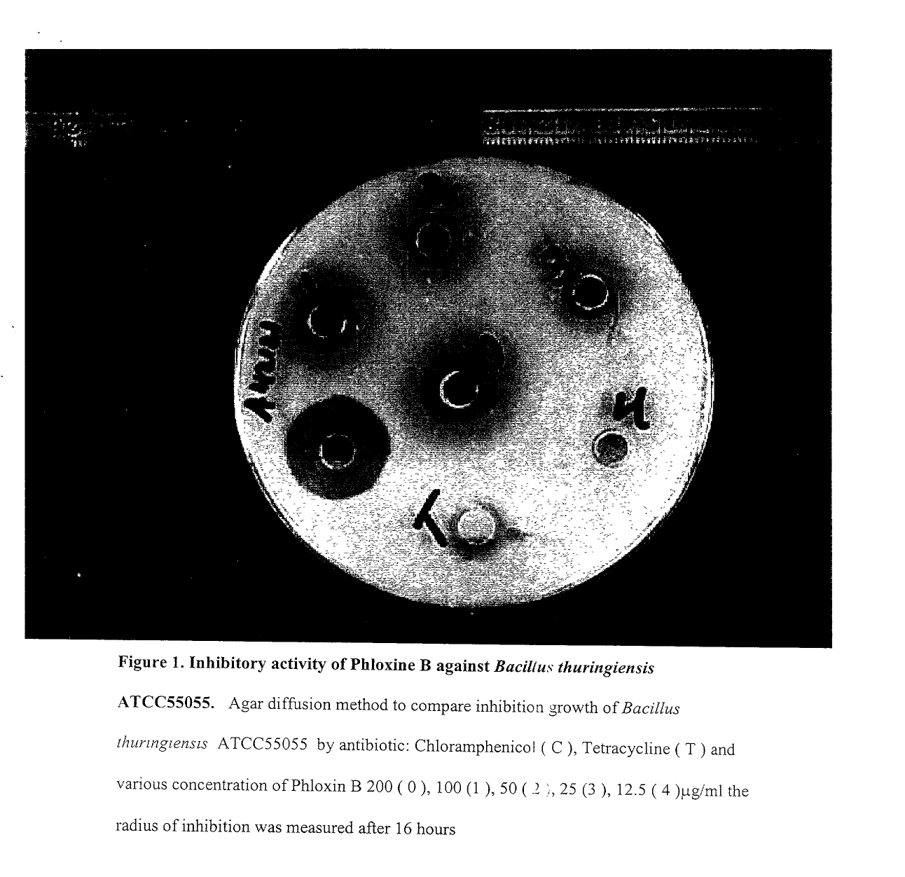

FIG. 2. Agar diffusion method to compare inhibition growth of Bacillus subtilis 1270 by antibiotic: Chloramphenicol, Tetracycline and various concentration of Phloxin B (200, 100, 50, 25, 12.5 μg/ml).

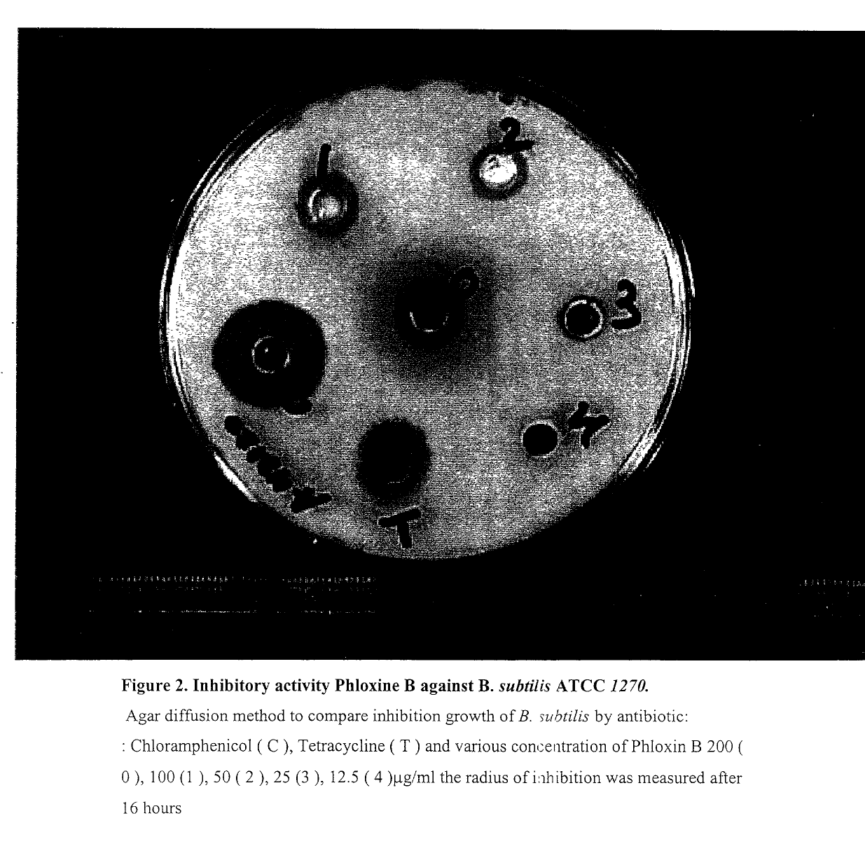

FIG. 3. Graphically represent the results of test to assess the ability of Phloxin B to inhibit growth of Bacillus thuringiensis ATCC55055 in mid-log phase culture and the survival rate that was measured by Colony-Forming Units (CFU).

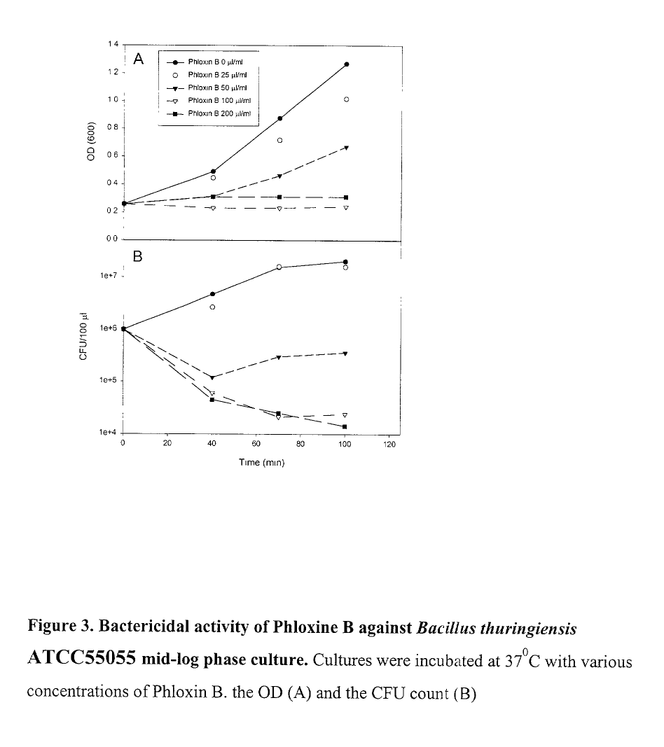

FIG. 4. The ability of various concentration of Phloxin B to inhibit growth of Bacillus subtilis 1270 in mid-log phase culture in deferent times.

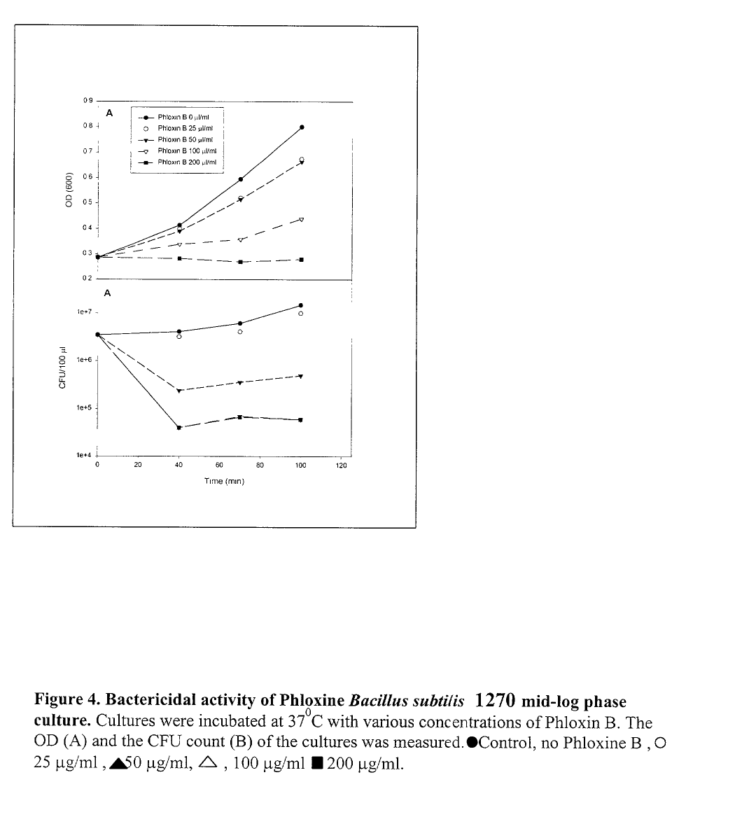

FIG. 5. The ability of various concentration of Phloxin B to inhibit growth of B. mycoides, B. thuringiensis and B. subtilis in mid-log phase culture after 100 mints of incubation.

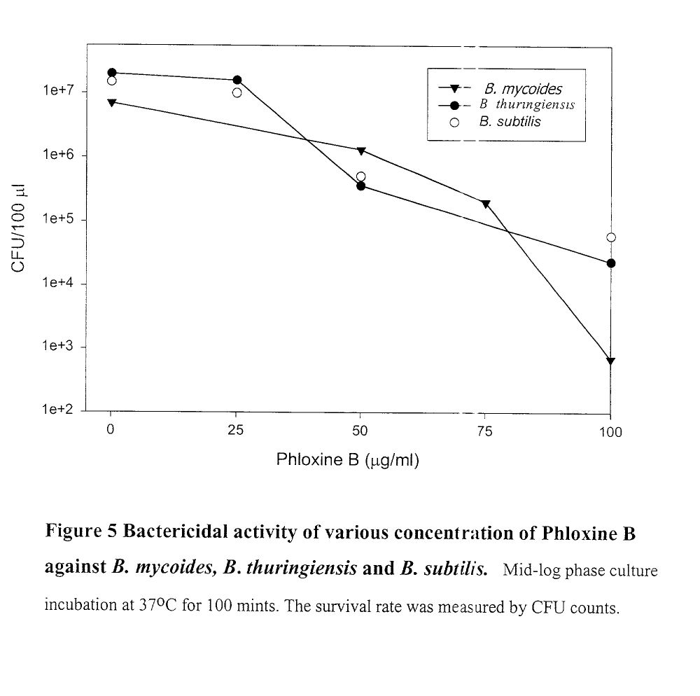

I claim:

1. A method of treating a subject suffering from infection with bacteria, with Phloxine B (2′,4′,5′,7′-tetrachlorofluorecein disodium salt) C₂₀H₂Br₄Cl₄O₅Na₂ also referred to as D&C red No. 28 that has a molecular weight of 829.6

2. Phloxine B can be used as antibiotic in treatment of infection caused by gram-positive bacteria.

3. Phloxine B can be used as antibiotic agent against gram-negative bacteria.

4. Phloxine B can be used as antibiotic agent against Bacillus spp.

5. The administration of Phloxine B and pharmaceutically acceptable diluent, adjuvant, carrier or agonists or light my allows reduction in Phloxine B concentration.

6. Effective dose rang from 10 μg/ml-20 μg/ml.

7. The medication may be in the from of unit dose in solid, semi-solid and infusible solution.
